(19) United States
(12) Patent Application Publication
(10) Pub. No.: US 2025/0000340 A1
(43) Pub. Date: Jan. 2, 2025

(54) SOFT FLEXIBLE LAPAROSCOPIC TROCAR

(71) Applicant: KING FAISAL UNIVERSITY, AL-AHSA (SA)

(72) Inventor: ABDULRAHMAN SALEH ALMULHIM, AL-AHSA (SA)

(21) Appl. No.: 18/216,682

(22) Filed: Jun. 30, 2023

(51) Int. Cl. A61B 1/00; A61B 1/313

(52) U.S. Cl. CPC A61B 1/00154; A61B 1/3132; A61B 2017/3429

(57) ABSTRACT

A method and device for providing a flexible and soft surgical trocar for use in laparoscopic procedures.

### BACKGROUND

### 1. Field

The present disclosure relates generally to an apparatus and method for providing percutaneous access to an internal operative site during a surgical procedure using a laparoscope. More particularly, the present disclosure relates to an access system which can be percutaneously introduced in a narrow diameter configuration and thereafter be bent, steered, or manipulated to accommodate passage of surgical instruments to hard to reach spaces within the surgical operating field.

### 2. Description of the Related Art

Minimally invasive surgical procedures involve surgically accessing an internal surgical site with small diameter tubes (typically 5 mm to 12 mm), usually referred to as trocars, which penetrate the skin and permit access to the desired surgical site. A viewing scope is introduced through one such trocar, and the surgeon operates using instruments introduced through the other appropriately placed trocars while viewing the operative site on a video monitor connected to the viewing scope. The surgeon is thus able to perform a wide variety of surgical procedures requiring only several 5 mm to 12 mm punctures at the surgical site. Patient trauma and recovery time are thus greatly reduced.

In this scenario, the design of suitable trocars must fulfill many requirements, particularly for those used in laparoscopic procedures. Trocars should be introducible within the patient with minimum trauma and with a minimal risk of injury to internal organs. The trocars used in laparoscopic procedures should be readily bendable, steerable, or capable of being manipulated, especially when used in minimally invasive procedures where the interventional medical device can curve or loop through the anatomy, making advancement of the device difficult. Unfortunately, however, to date most trocars are stiff and flexible to permit penetration through the skin.

What is needed is a method and device that can provide safe, efficient, and precise access to otherwise difficult to reach anatomical locations such as minimally invasive surgical procedures involving laparoscopes satisfying these needs.

### SUMMARY

The present subject matter is directed towards a method and device for providing a flexible and soft surgical trocar for use in laparoscopic procedures.

In a first aspect of the present subject matter, a soft and flexible trocar is provided, the soft flexible trocar comprising: a handle; a trocar shaft extending from the cylindrical handle where the cylindrical handle and trocar shaft define a longitudinal axis which extends through and along the length of the device and has an axial lumen. The trocar shaft comprises a flexible material adapted to be bent, steered, or manipulated to exhibit a curvature off of the longitudinal axis. The trocar shaft defines an oblique end surface. The trocar shaft includes a mounting element mounted therewithin such that the mounting element attaches to the handle. The cylindrical handle also includes a seal element mounted therewithin defining an aperture for a sealed reception of the elongate object of the trocar shaft. In certain embodiments, the flexible material of the trocar is a cannula.

In this regard, the present subject matter relates to a soft flexible surgical trocar for use with a laparoscope, comprising: a cylindrical handle with a hole traversing from a first side of the cylindrical handle to a second side of the cylindrical handle; a mounting element located adjacent the hole on the second side of the cylindrical handle; and a trocar shaft connected to the mounting element and which extends away from the hole on the second side of the cylindrical handle, wherein the trocar shaft is flexible and capable of being bent at an angle with respect to a longitudinal axis of said soft flexible surgical trocar, and wherein the trocar shaft terminates in an axial lumen.

In another embodiment, the present subject matter relates to a soft flexible surgical trocar cannula for use with a laparoscope, comprising: a cylindrical handle with a hole traversing from a first side of the cylindrical handle to a second side of the cylindrical handle; a mounting element located adjacent the hole on the second side of the cylindrical handle; and a cannula sleeve connected to the mounting element and which extends away from the hole on the second side of the cylindrical handle, wherein said cannula sleeve is flexible and capable of being bent at an angle with respect to a longitudinal axis of said soft flexible surgical trocar cannula, and wherein the cannula sleeve terminates in an axial lumen.

A second aspect of the present subject matter is a method which makes use of the soft flexible trocar for bending, flexing, or steering the trocar to access difficult to reach anatomical surgical sites.

In this regard, an embodiment of the present subject matter is directed to a method which uses a soft flexible surgical trocar with laparoscopes in a surgical procedure, the method comprising: positioning a soft flexible trocar shaft near a surgical site; rotating a cylindrical handle connected to said soft flexible trocar shaft in a clockwise fashion to bend the soft flexible trocar shaft; and controlling the bending of the soft flexible trocar shaft at an angle with respect to a longitudinal axis of said soft flexible surgical trocar, wherein said angle with respect to said longitudinal axis of said soft flexible surgical trocar is between about 5 and about 40 degrees.

These and other features of the present subject matter will become readily apparent upon further review of the following specification.

### BRIEF DESCRIPTION OF THE DRAWINGS

FIG. 1 is a diagram of the present surgical trocar.

FIG. 2 is a diagram of a surgical trocar cannula used with the present surgical trocar.

Similar reference characters denote corresponding features consistently throughout the attached drawings.

### DETAILED DESCRIPTION OF EMBODIMENTS

The following detailed description is merely exemplary in nature and is not intended to limit the described embodiments or the application and uses of the described embodiments. Any implementation described herein with the words “exemplary” or “illustrative” is not necessarily construed as preferred or advantageous over other implementations. All of the implementations described below are exemplary implementations provided to enable persons skilled in the art to make or use the embodiments of the disclosure and are not intended to limit the scope of the disclosure, which is defined by the claims. For the purposes of the description herein, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed therein are not to be considered as limiting, unless the claims expressly state otherwise.

A soft and flexible trocar is provided, the soft flexible trocar comprising: a handle; a trocar shaft extending from the cylindrical handle where the cylindrical handle and trocar shaft define a longitudinal axis which extends through and along the length of the device and has an axial lumen. The trocar shaft comprises a flexible material adapted to be bent, steered, or manipulated to exhibit a curvature off of the longitudinal axis. The trocar shaft defines an oblique end surface. The trocar shaft includes a mounting element mounted therewithin such that the mounting element attaches to the handle. The cylindrical handle also includes a seal element mounted therewithin defining an aperture for a sealed reception of the elongate object of the trocar shaft. In certain embodiments, the flexible material of the trocar is a cannula.

The use of a soft flexible surgical trocar as described herein, as opposed to currently known trocars made of a rigid material, is a critical aspect of the present subject matter. The soft, flexible material permits the present trocar to be used not only in typical laparoscopic procedures, but also in situations where it has been historically difficult to use previous trocars, including by way of non-limiting example, in obese patients, in patients with previous open abdominal surgery, and in small abdominal cavities, such as, for example, in a child. The result is the ability to use more laparoscopic instruments, and to manipulate such instruments, during difficult laparoscopic procedures while minimizing the risk of the trocar being broken, as is the case with the current rigid trocars.

In this regard, the present subject matter relates to a soft flexible surgical trocar for use with a laparoscope, comprising: a cylindrical handle with a hole traversing from a first side of the cylindrical handle to a second side of the cylindrical handle; a mounting element located adjacent the hole on the second side of the cylindrical handle; and a trocar shaft connected to the mounting element and which extends away from the hole on the second side of the cylindrical handle, wherein the trocar shaft is flexible and capable of being bent at an angle with respect to a longitudinal axis of said soft flexible surgical trocar, and wherein the trocar shaft terminates in an axial lumen.

In certain embodiments, the angle with respect to the longitudinal axis of the soft flexible surgical trocar can be between about 5 and about 40 degrees. In further embodiments, the cylindrical handle can be turned counterclockwise to cause the trocar shaft to bend at the angle with respect to the longitudinal axis of the soft flexible surgical trocar.

In an embodiment, the axial lumen of the trocar shaft can terminate in an inclined surface obliquely arranged relative to the longitudinal axis of the trocar shaft such that the inclined surface facilitates a passage of the trocar shaft through tissue. In this regard, the trocar shaft can mounted to the hole of the second side of the cylindrical handle using the mounting element.

In another embodiment, the present subject matter relates to a soft flexible surgical trocar cannula for use with a laparoscope, comprising: a cylindrical handle with a hole traversing from a first side of the cylindrical handle to a second side of the cylindrical handle; a mounting element located adjacent the hole on the second side of the cylindrical handle; and a cannula sleeve connected to the mounting element and which extends away from the hole on the second side of the cylindrical handle, wherein said cannula sleeve is flexible and capable of being bent at an angle with respect to a longitudinal axis of said soft flexible surgical trocar cannula, and wherein the cannula sleeve terminates in an axial lumen.

In an embodiment of the trocar cannula, the cannula sleeve can have a soft flexible part capable of being bent and a solid part. Similarly, the trocar cannula can have a soft flexible part capable of being bent and a solid part.

In another embodiment, the angle with respect to the longitudinal axis of the soft flexible surgical trocar cannula can be between about 5 and about 40 degrees. In this regard, the cylindrical handle can be turned counterclockwise to cause the cannula sleeve to bend at the angle with respect to the longitudinal axis of the soft flexible surgical trocar cannula.

In a further embodiment, the cannula sleeve can be mounted to the hole of the second side of the cylindrical handle using the mounting element.

In this regard, an embodiment of the present subject matter is directed to a method which uses a soft flexible surgical trocar with laparoscopes in a surgical procedure, the method comprising: positioning a soft flexible trocar shaft near a surgical site; rotating a cylindrical handle connected to said soft flexible trocar shaft in a clockwise fashion to bend the soft flexible trocar shaft; and controlling the bending of the soft flexible trocar shaft at an angle with respect to a longitudinal axis of said soft flexible surgical trocar, wherein said angle with respect to said longitudinal axis of said soft flexible surgical trocar is between about 5 and about 40 degrees.

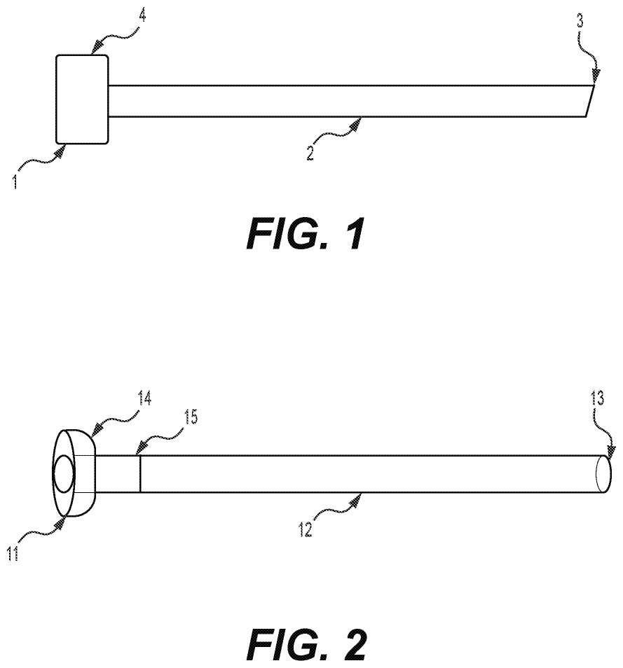

As shown in FIG. 1, in an exemplary embodiment, a soft and flexible trocar is provided. The soft flexible trocar comprises a cylindrical handle (1) which provides an input for the surgeon to guide the ingress of the trocar shaft (2) into the surgical space. The trocar shaft (2) extends from the cylindrical handle (1) where the cylindrical handle (1) and trocar shaft (2) define a longitudinal axis which extends through and along the length of the device and has an axial lumen (3). The trocar shaft (2) comprises a flexible material adapted to be bent, steered, or manipulated to exhibit a curvature off of the longitudinal axis. The trocar shaft (2) defines an oblique end surface. The cylindrical handle (1) includes a mounting element (4) mounted therewithin such that the mounting element (4) attaches the trocar shaft to the cylindrical handle (1). The cylindrical handle (1) also includes a seal element mounted therewithin defining an aperture for a sealed reception of the elongate object of the trocar shaft (2). The other end of the cylindrical handle (1) includes a hole into which a laparoscope or surgical instrument may be inserted and guided through the trocar shaft towards the axial lumen (3) where it would exit therefrom into the surgical space. The rotation of the cylindrical handle in a clockwise fashion about the longitudinal axis of the trocar shaft can cause the trocar shaft to bend in an angle of about 5 to about 40 degrees with respect to the longitudinal axis of the trocar shaft. The axial lumen (3) of the trocar shaft terminates in an inclined surface obliquely arranged relative to the longitudinal axis of the trocar shaft. This surface facilitates the passage of the trocar shaft through the tissue. The trocar shaft may be fabricated from any material which is capable of receiving the assembly of a dilator, surgical instrument, or laparoscope. The materials are desirably medical grade materials including polymers and metals.

As shown in FIG. 2, in an exemplary embodiment, a soft and flexible trocar cannula is depicted where it includes cannula cylindrical handle (11), and a cannula sleeve include a soft, flexible part (12) and a rigid part (15) where the soft, flexible part can be bent in a like manner as discussed above with regards to FIG. 1. In this instance, the counterclockwise rotation of the cannula cylindrical handle (11) causes the soft, flexible part of the cannula sleeve of the trocar cannula to bend in an angle of about 5 to about 40 degrees with respect to the longitudinal axis of the cannula sleeve. It is noted that the axial lumen (13) of the cannula sleeve is perpendicular to the longitudinal axis of the cannula sleeve of the trocar cannula and is not inclined as with the trocar shaft of FIG. 1. The trocar cannula also includes a mounting element (14) within the cannula cylindrical handle (11) which functions to attach the rigid part (15) of the cannula sleeve to the cannula cylindrical handle (11). The cannula cylindrical handle (11) also includes a seal element mounted therewithin defining an aperture for a sealed reception of the elongate object of the trocar cannula sleeve. The trocar cannula sleeve may be fabricated from any material which is capable of receiving the assembly of a surgical instrument or laparoscope. The materials are desirably medical grade materials including polymers and metals.

The method includes using the soft flexible surgical trocar for use with laparoscopes in a surgical procedure and includes the steps of positioning soft flexible trocar shaft near the surgical site; rotating a cylindrical handle connected to said the soft flexible trocar shaft in a clockwise fashion to bend the soft flexible trocar shaft; and controlling the bending of the soft flexible trocar shaft at an angle with respect to a longitudinal axis of said soft flexible surgical trocar.

It is to be understood that the soft flexible surgical trocar for use with laparoscopes and the associated method is not limited to the specific embodiments described above but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A soft flexible surgical trocar configured for use with a laparoscope, comprising:
a cylindrical handle with a hole traversing from a first side of the cylindrical handle to a second side of the cylindrical handle;
a mounting element located adjacent the hole on the second side of the cylindrical handle;
a trocar shaft connected to the mounting element and which extends away from the hole on the second side of the cylindrical handle; and
a seal element mounted within the cylindrical handle defining an aperture for a sealed reception of the trocar shaft,
wherein the trocar shaft is flexible and capable of being bent at an angle with respect to a longitudinal axis of said soft flexible surgical trocar, and wherein the trocar shaft terminates in an axial lumen,
wherein said cylindrical handle is turned clockwise to cause said trocar shaft to bend at said angle with respect to said longitudinal axis of said soft flexible surgical trocar, and
wherein when said soft flexible surgical trocar is used with a surgical trocar cannula said cylindrical handle is turned counterclockwise to cause said surgical trocar cannula to bend at a second angle with respect to a longitudinal axis of said soft flexible surgical trocar.

2. The soft flexible surgical trocar for use with a laparoscope as recited in claim 1, wherein said angle with respect to said longitudinal axis of said soft flexible surgical trocar is between about 5 and about 40 degrees.

3. (canceled)

4. The soft flexible surgical trocar for use with a laparoscope as recited in claim 2, wherein said axial lumen of said trocar shaft terminates in an inclined surface obliquely arranged relative to said longitudinal axis of said trocar shaft such that said inclined surface facilitates a passage of said trocar shaft through tissue.

5. (canceled)

6. A soft flexible surgical trocar cannula for use with a laparoscope, comprising:
a cylindrical handle with a hole traversing from a first side of the cylindrical handle to a second side of the cylindrical handle;
a mounting element located adjacent the hole on the second side of the cylindrical handle; and
a cannula sleeve connected to the mounting element and which extends away from the hole on the second side of the cylindrical handle,
wherein said cannula sleeve is flexible and capable of being bent at an angle with respect to a longitudinal axis of said soft flexible surgical trocar cannula, and wherein the cannula sleeve terminates in an axial lumen.

7. The soft flexible surgical trocar cannula for use with a laparoscope, as recited in claim 6, wherein said cannula sleeve has a soft flexible part capable of being bent and a solid part.

8. The soft flexible surgical trocar cannula for use with a laparoscope, as recited in claim 7, wherein said trocar cannula has a soft flexible part capable of being bent and a solid part.

9. The soft flexible surgical trocar cannula for use with a laparoscope, as recited in claim 8, wherein said angle with respect to said longitudinal axis of said soft flexible surgical trocar cannula is between about 5 and about 40 degrees.

10. The soft flexible surgical trocar cannula for use with a laparoscope, as recited in claim 9, wherein said cannula sleeve is mounted to said hole of said second side of said cylindrical handle using said mounting element.

11. The soft flexible surgical trocar cannula for use with a laparoscope, as recited in claim 6, wherein said cylindrical handle is turned counterclockwise to cause said cannula sleeve to bend at said angle with respect to said longitudinal axis of said soft flexible surgical trocar cannula.

12. A method which uses a soft flexible surgical trocar with laparoscopes in a surgical procedure, the method comprising:
positioning a soft flexible trocar shaft near a surgical site;
rotating a cylindrical handle connected to said soft flexible trocar shaft in a clockwise fashion to bend the soft flexible trocar shaft; and
controlling the bending of the soft flexible trocar shaft at an angle with respect to a longitudinal axis of said soft flexible surgical trocar, wherein said angle with respect to said longitudinal axis of said soft flexible surgical trocar is between about 5 and about 40 degrees.
